(19) United States
(12) Patent Application Publication
(10) Pub. No.: US 2005/0288655 A1
(43) Pub. Date: Dec. 29, 2005

(54) Laser fiber for endovenous therapy having a shielded distal tip

(76) Inventors: Howard Root, Excelsior, MN (US); Jim Kauphusman, Champlin, MN (US); Nancy Arnold, Andover, MN (US); John VanScoy, Shakopee, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/879,701

(22) Filed: Jun. 29, 2004

(51) Int. Cl. A61B 18/22

(52) U.S. Cl. 606/15

(57) ABSTRACT

An endovenous laser fiber optic member for endovenous laser therapy of peripheral veins of the body including a flexible heat resistant tip shield covering the distal end of the laser fiber optic. The tip shield has an irregular surface to increase ultrasound reflectivity. The tip shield also improves deflectability of the distal end and acts as a heat sink and heat energy dissipater.

### FIELD OF THE INVENTION

The present invention relates generally to the field of surgical instruments utilizing light application via optical fibers placed within the body. More particularly, the present invention relates to endovenous laser therapy of the peripheral veins, such as greater saphenous veins of the leg, for treatment of varicose veins.

### BACKGROUND OF THE INVENTION

Varicose veins are enlarged, tortuous, often blue in color and commonly occur in the legs below the knee. Varicose veins are the most common peripheral vascular abnormality affecting the legs in the United States. Varicose veins often lead to symptomatic venous insufficiency. Greater saphenous vein reflux is the most common form of venous insufficiency in symptomatic patients and is frequently responsible for varicose veins in the lower leg. This occurs in about 25% of women and about 15% of men.

All veins in the human body have valves that open to allow the flow of blood toward the heart and close to prevent backflow of blood toward the extremities. The backflow of blood is also known as reflux. The venous check valves perform their most important function in the veins of the legs where venous return flow is most affected by gravity. When the venous valves fail to function properly, blood leaks through the valves in a direction away from the heart and flows down the leg in the wrong direction. The blood then pools in the superficial veins under the skin resulting in the bulging appearance typically seen in varicose veins. The pooling of blood in the leg veins tends to stretch the thin elastic walls of the veins, which in turn causes greater disruption in the function of the valves, leading to worsening of the varicosities. When varicose veins become severe, the condition is referred to as chronic venous insufficiency. Chronic venous insufficiency can contribute to the development of pain, swelling, recurring inflammation, leg ulcers, hemorrhage and deep vein thrombosis.

Traditionally, varicose veins have been treated by a surgical procedure known as stripping. In stripping, varicose veins are ligated and completely removed. More recently, varicose veins have been treated by endovenous laser therapy. Endovenous laser therapy treats varicose veins of the leg by eliminating the highest point at which blood flows back down the veins, thereby cutting off the incompetent venous segment. Endovenous laser therapy has significant advantages over surgical ligation and stripping. In general, endovenous laser therapy has reduced risks related to anesthesia, less likelihood of surgical complications, reduced costs and a shorter recovery period than ligation and stripping.

Endovenous laser therapy involves the use of a bare tipped laser fiber to deliver laser energy to the venous wall from within the vein lumen that causes thermal vein wall damage at the desired location. The subsequent fibrosis at this location results in occlusion of the vein that prevents blood from flowing back down the vein. Generally, endovenous laser therapy utilizes an 810 to 980 nanometer diode laser as a source of laser energy that is delivered to the venous wall in a continuous mode with a power of about 10 to 15 Watts.

An exemplary endovenous laser therapy procedure is disclosed in U.S. Pat. No. 4,564,011 issued to Goldman. The Goldman patent discloses the use of an optical fiber to transmit laser energy into or adjacent to a blood vessel to cause clotting of blood within the vessel or to cause scarring and shrinkage of the blood vessel.

A typical endovenous laser therapy procedure includes the location and mapping of venous segments with duplex ultrasound. An introducer sheath is inserted into the greater saphenous vein over a guide wire, followed by a bare tipped laser fiber about 600 micrometers in diameter. The bare distal end of the laser fiber is advanced to within 1 to 2 cm of the sapheno-femoral junction. Laser energy is then applied at a power level of about 10 to 15 watts along the course of the greater saphenous vein as the laser fiber is slowly withdrawn. Generally, positioning of the laser fiber is done under ultrasound guidance and confirmed by visualization of the red aiming beam of the laser fiber through the skin. The application of laser energy into the vein utilizes the hemoglobin in red blood cells as a chromophore. The absorption of laser energy by hemoglobin heats the blood to boiling, producing steam bubbles which cause full thickness thermal injury to the vein wall. This injury destroys the local venous endothelium and creates a full-length thrombotic occlusion of the greater saphenous vein. An example of current techniques for endovenous laser therapy procedures is described in U.S. Patent Publ. No. 2003/0078569 A1, the disclosure of which is hereby incorporated by reference.

While current endovenous laser therapy procedures offer a number of advantages over conventional ligation and stripping, challenges remain in successfully implementing an endovenous laser therapy procedure. The accurate localization of the bare distal end of the laser fiber can be difficult even with ultrasound assistance. In addition, the bare distal end of the laser fiber is transparent to fluoroscopy. Because of the relatively small diameter and sharpness of the laser fiber, the distal tip of the laser fiber can sometimes enter or puncture and exit the vein wall while the laser fiber is being advanced up a tortuous greater saphenous vein. Laser fibers used in current endovenous laser therapy procedures are glass optical fibers coaxially surrounded by protective plastic jacket or coating. When this plastic jacket is exposed to heat during the endovenous therapy procedure, the plastic jacket tends to melt or burn back from the distal tip of the fiber as the procedure is performed leaving undesirable foreign matter in the vein. In addition, it is possible for the tip of the laser fiber to come into close contact with the venous wall during the endovenous laser treatment. When this occurs there is an increased possibility of perforation of the venous wall due to the unintended localized application of laser energy and the consequent generation of heat. This can lead to additional complications in the endovenous procedure.

### SUMMARY OF THE INVENTION

The present invention is an endovenous laser fiber that includes a flexible heat resistant tip shield coaxially surrounding the distal end of the laser fiber and having an irregular surface. The tip shield may take the form of a coil spring, coiled wire or a slotted tube that has a rounded or chamfered distal most end. Preferably, the distal tip shield is formed of a flexible spring formed of stainless steel, platinum-iridium alloy or nitinol that coaxially surrounds the distal portion of the laser fiber transverse to the longitudinal axis of the laser fiber while leaving at least the distal end face exposed.

The spring coil tip shield substantially increases the visibility of the laser fiber tip to ultrasound because of the increased ultrasound reflectivity. The tip shield also makes the fiber end visible to fluoroscopy when it is made from radio-opaque material. In addition, the tip shield protects the laser fiber from damage and deflects the laser fiber tip from digging into the vein wall during as it is advanced into the vein. The tip shield may be generally cylindrical or tapered in shape. Because the spring coil tip shield tends to deflect the laser fiber tip from the vein wall, the risk of inadvertent application of laser energy directly into the venous wall is also reduced, thereby decreasing the risk of inadvertent venous wall perforation.

The spring coil tip shield also acts as a heat sink absorbing excess heat generated in the proximity of the distal end of the laser fiber and improving heat dissipation at the distal tip of the laser fiber. Improved heat dissipation and the associated set back provided by the tip shield reduces the potential for burn back of the plastic jacket around the laser fiber and improves heat transfer from the optical fiber to the blood and other surrounding tissue. The improved heat transfer from the spring coil tip shield tends to encourage the clotting of blood in the blood vessel, thus improving results in endovenous laser therapy procedures.

### BRIEF DESCRIPTION OF THE DRAWINGS

FIG. 1 is a perspective view of the tip of an embodiment of the laser fiber of the present invention;

FIG. 2 is an exploded perspective view of the laser fiber of FIG. 1;

FIG. 3 is a plan view of the laser fiber;

FIG. 4 is an exploded plan view of the laser fiber;

FIG. 5 is a graph of deflection comparing the present invention to laser fibers without a tip shield;

FIG. 6 is a detailed cross-sectional view of an embodiment of laser fiber tip;

FIG. 7 is a schematic view of the entire length of the laser fiber;

FIG. 8 is a plan view of another embodiment of the laser fiber;

FIG. 9 is a cross-sectional view of the laser fiber of FIG. 8 taken at section line 9-9; and

FIG. 10 is a detailed cross-sectional view of the laser fiber depicted in FIG. 9.

### DETAILED DESCRIPTION OF THE PREFERRED EMBODIMENTS

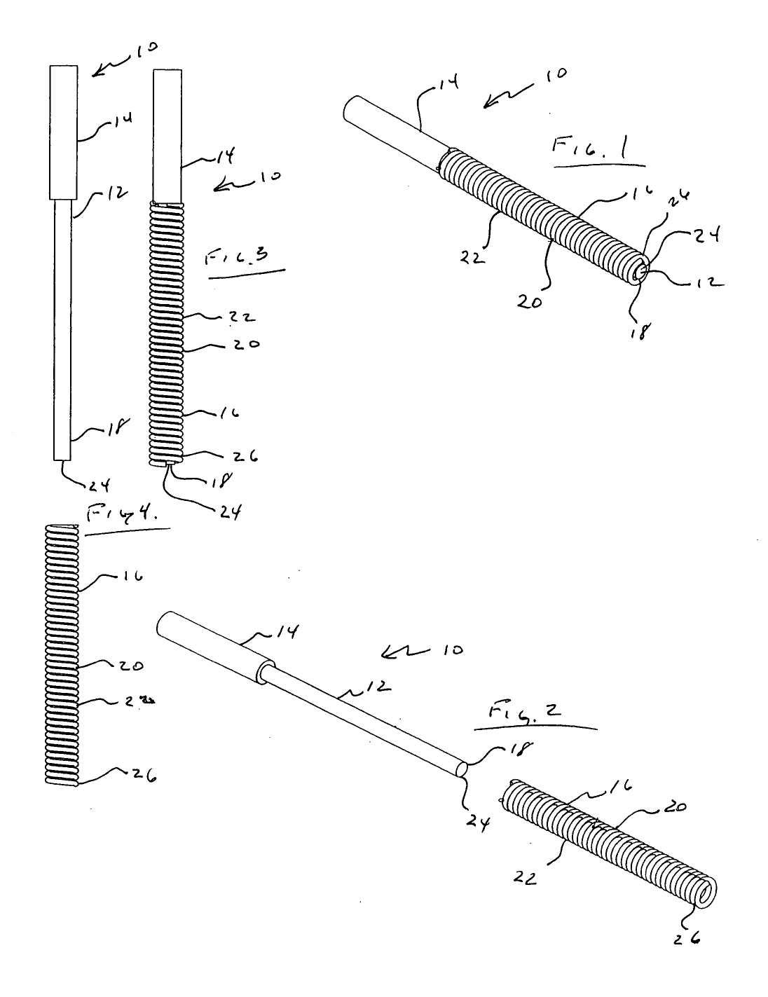

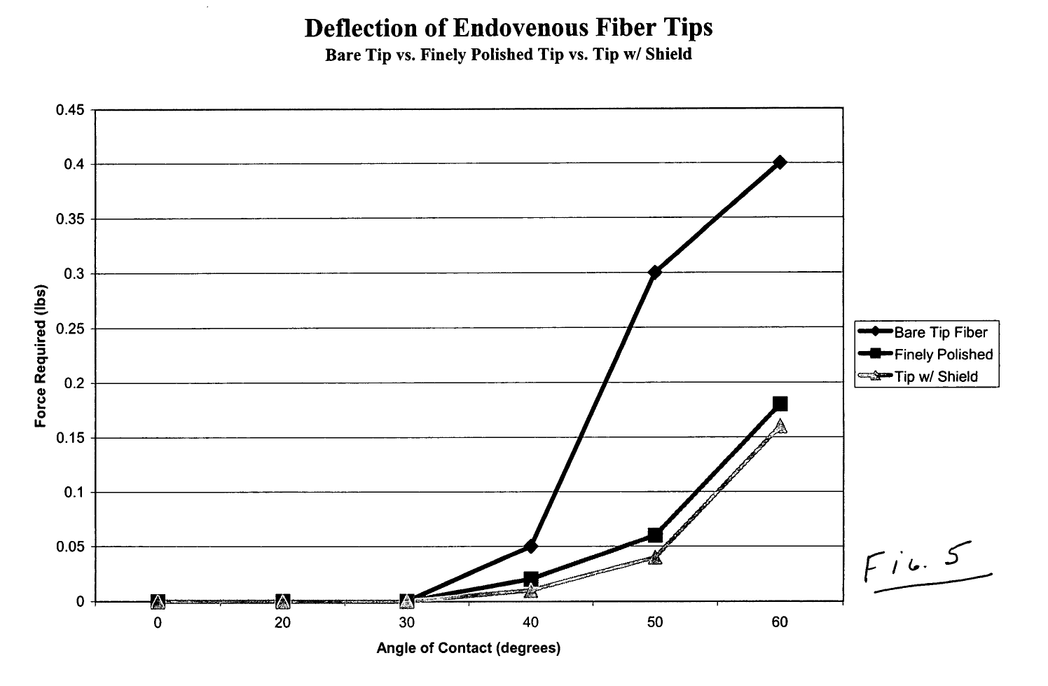

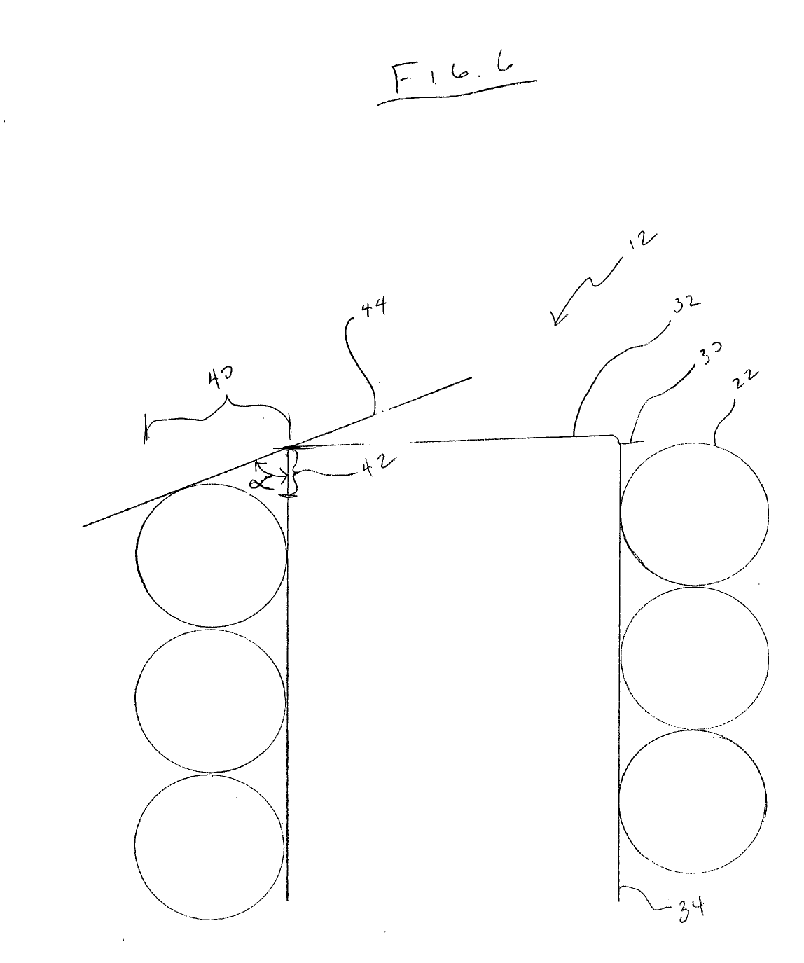

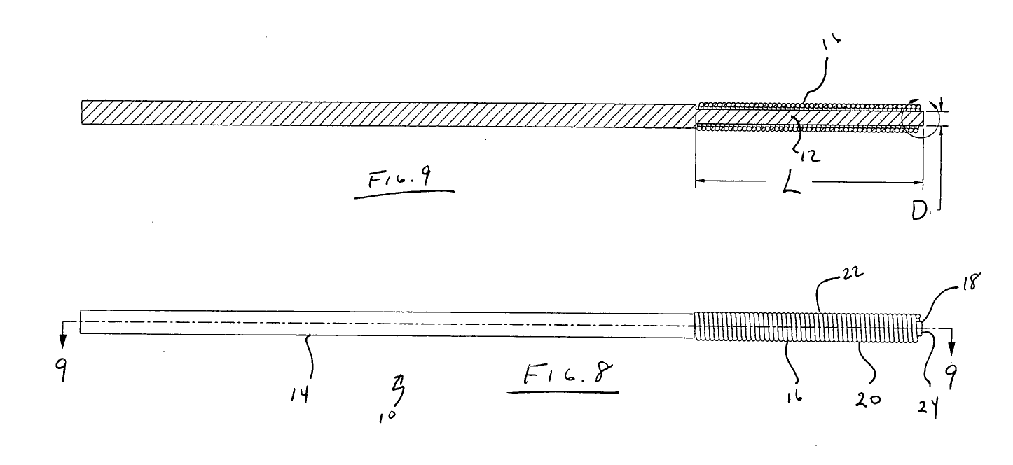

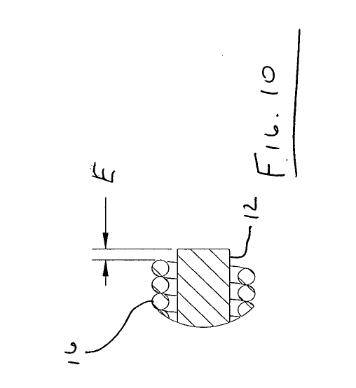

Referring to FIGS. 1-7, the endovenous laser fiber 10 of the present invention generally includes an optic fiber 12 coaxially surrounded by a protective jacket 14 and having at a distal portion 18 a flexible heat resistant tip shield 16 that has an irregular surface.

Optic fiber 12 is desirably a 400 to 600 micron glass optical fiber with a finely polished distal tip end, although a polymer fiber could be used. Those skilled in the art will understand that the designated dimension of the glass optical fiber refers to the diameter D of the fiber including the core and cladding but exclusive of the protective jacket 14. The exterior dimensions of the protective jacket are larger. While a single optical fiber is described, it will be recognized that optic fiber 12 could also comprise a stranded arrangement of multiple optical fibers. Desirably the endovenous laser fiber 10 is about three and one half meters long. The optic fiber 12 is preferably provided with a standardized connector 17, such as an SMA-905 standard connector with an adjustable fiber lock, for connection to a laser source console (not shown). The laser source console is preferably a solid state diode laser console operating at a wavelength of 810 nanometers, 940 nanometers or 980 nanometers and supporting a maximum power output of about 15 Watts.

Optic fiber 12 is coaxially surrounded by a protective jacket 14. Protective jacket 14 is generally conventional and is desirably formed of a biocompatible plastic material. Protective jacket 14 preferably covers substantially the entire longitudinal length of optic fiber 12 leaving exposed length L approximately ½ to 2 cm at the distal portion 18 of the optic fiber 12. At least a portion of this exposed distal end 18 is covered by a rigid or flexible heat resistant tip shield 16.

Heat resistant tip shield 16 preferably covers the entire exposed distal portion 18 of optic fiber 12. Tip shield 16 coaxially surrounds the distal portion 18 of the optic fiber 12 transverse to the longitudinal axis of the optic fiber 12 while leaving the distal tip face 24 exposed. Tip shield 16 is formed of a rigid or flexible, heat resistant, heat conductive material having an irregular ultrasound reflective surface. Tip shield 16 also is desirably, readily deflectable upon encountering an obstruction at an acute angle.

In a preferred embodiment, tip shield 16 is desirably formed of a stainless steel, platinum/iridium or nitinol coil spring 22 tightly wound about the distal portion 18 of optic fiber 12. Coil spring 22 is desirably about ½ to 2 cm in length and has an outside diameter of approximately 950 to 1100 μl. Coil spring 22 is desirably formed from heat resistant, thermally conductive wire 24. Wire 24 is desirably stainless steel having a wire diameter of between 100 and 230 μl. Tip shield 16 can also be formed from stainless steel, platinum/iridium or nitinol in the form of a slotted tube rather than a coil spring. Desirably coil spring 22 is of such a length and position on the distal portion 18 of optic fiber 12 so that tip end 24 of optic fiber 12 is substantially aligned with the distal end 26 of coil spring 22. Tip shield 16 may be cylindrical or tapering. Tip shield 16 may be secured to glass fiber 12 with a high temperature adhesive.

Alternately, the tip end 24 of optic fiber 12 may extend slightly beyond distal end 26 of coil spring 22. For example, tip end 24 may extend beyond the termination of tip shield 16 a distance E of about 0.003 inches. Tip end 24 may be rounded in shape or any other shape but is preferably planar and forms a ninety-degree angle with the long axis of the optic fiber 12.

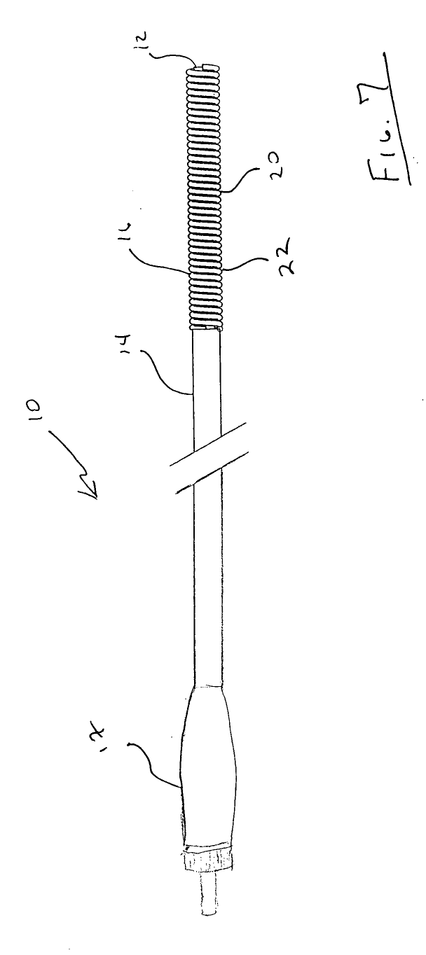

In a preferred embodiment, as shown in the cross-sectional detail of FIG. 6, the tip end 24 of optic fiber 12 is surfaced to a substantially flat shape and includes a relatively sharp circumference 30 at the boundary between the substantially flat shape 32 of tip end 24 and the side wall 34 of the optic fiber 12. It is believed that the circumference 30 of tip end 24 is primarily responsible for the snagging or catching of the tip end 24 along the interior wall of the blood vessel. In order to minimize the potential for snagging or catching of the tip end 24, the effective transverse thickness 40 of tip shield 16 and the longitudinal offset position 42 of the tip shield 16 relative to the circumference 30 are dimensioned such that a line 44 tangent to tip shield 16 will intersect the circumference 30 assuming that the optic fiber meets the vessel wall at an angle alpha not greater than sixty degrees.

Tip shield 16 is formed of a heat resistant, heat conductive material. Tip shield 16 is desirably substantially flexible as well. Tip shield 16 desirably preferably should withstand temperatures up to approximately 1000° F. For example, tip shield 16 desirably is formed of a material having a thermal conductivity of at least 12 W/mK at 273 Kelvin.

Referring to FIG. 5, test results demonstrate that the optic fiber 12 with tip shield 16 demonstrates improved deflection qualities as compared to a bare tipped fiber and a finely polished tip fiber. The optic fiber 12 with tip shield 16 requires approximately 0.020 pound less force application to be advanced at a wall contact angle of between 50 and 60 degrees than a polished tip fiber. As compared to a bare tip fiber the tip shielded optic fiber 12 requires about 0.250 pounds less force application at a contact angle of between 50 and 60 degrees.

Testing was performed in the following fashion. The tested fibers were advanced into a longitudinally halved PTFE tubular sheath representing a model of a blood vessel to contact the sheath wall at the indicated angles and the force required to the advance the fiber against the sheath was measured and recorded. The sheath utilized in the test procedure was of the type typically used in vascular introducers. PTFE material does not closely simulate the qualities of a blood vessel wall but is conventionally utilized for testing purposes because of its ready availability.

Endovenous laser fiber 10 is utilized with a conventional guide wire and introducer during the endovenous laser therapy process. Insertion and placement of endovenous laser fiber 10 is largely accomplished by conventional techniques.

In operation, an endovenous laser therapy procedure begins with a physical examination of the limb to be treated. Transverse measurements of the greater saphenous vein are made 2-3 cm below the sapheno-femoral junction and along the course of the greater saphenous vein with ultrasound and Doppler ultrasound. Doppler ultrasound can be utilized to confirm retrograde flow at the sapheno-femoral junction. Utilizing ultrasound, the location of the greater saphenous vein is recorded and an outline of the course of the greater saphenous is made on the leg with a marking pen. A desired insertion site for the catheter is also marked.

The limb to be treated is then prepped and draped in sterile fashion and the ultrasound transducer is enclosed in a sterile covering. The physician then cannulates the greater saphenous vein, typically using a 19-gauge needle, utilizing the Seldinger technique under ultrasound guidance. The physician should confirm the presence of non-pulsatile venous flow through the needle to confirm that the needle is in the vein. Next, the physician inserts a preferably 0.035 inch guide wire into the vessel and removes the needle over the guide wire. Next, the physician passes an introducer sheath over the guide wire and advances the introducer sheet into the sapheno-femoral junction. Preferably, a 5-French introducer sheath is used. The end of the sheath is desirably positioned at the proximal edge of the treatment area, generally 2-3 cm distal to the sapheno-femoral junction. The distal tip of the introducer sheath should not be positioned closer than 1 cm distal to the sapheno-femoral junction as this will place the fiber tip into the common femoral vein. The physician then removes the internal sheath dilator and guide wire and flushes the sheath with saline using standard technique.

The physician next prepares the laser console in accordance with its operating instructions and, outside of the sterile field, removes the cover from the laser fiber connector 17 and connects the laser fiber to the laser console and activates a red aiming beam. The physician inserts the endovenous laser fiber 10 into the introducer sheath and advances the laser fiber until a holder (not shown) snaps into the hub of the introducer sheath. The holder is designed so that the laser fiber 10 is exposed by approximately I cm beyond the distal tip of the introducer sheath. Next, utilizing ultrasound the physician confirms the position of the endovenous laser fiber 10 and the introducer sheath. The endovenous laser fiber 10 should be exposed slightly beyond the introducer sheath and located at least 2 cm distal to the sapheno-femoral junction. This is confirmed by visualization of the red aiming beam through the skin with the room lights extinguished.

The physician then administers a local anesthetic subcutaneously throughout the entire treatment area. While protecting the eyes of all operating room personal with laser safety glasses, the physician places the laser console in the ready mode, usually at 14 watts continuous power. With the lights turned down, the physician holds the introducer sheath by the hub, activates the laser and simultaneously withdraws the introducer sheath, desirably at a rate of about 2 mm per second. The introducer sheath desirably includes markings to aid in measurement during removal. Upon completion of the procedure along the entire treatment length, the laser is turned off, the introducer sheath is removed and the endovenous laser fiber 10 is removed from the vessel. Compression is applied to the wound until bleeding stops and a hemostatic bandage is applied over the percutaneous puncture.

The present invention may be embodied in other specific form without departing from the spirit of the essential attributes thereof, therefore, the illustrated embodiment should be considered in all respect as illustrative and not restrictive, reference being made to the appended claims rather than to the foregoing description to indicate the scope of the invention.

1. An laser fiber for endovenous laser therapy of veins, the laser fiber comprising:
a fiber optic member having a proximal end and a distal end, the proximal end including a connector adapted to connect to a therapeutic laser and the distal end adapted to emit laser energy when the fiber optic member is connected to the therapeutic laser;
a protective jacket coaxially covering substantially an entire length of the fiber optic member while leaving a distal portion proximate the distal end of the fiber optic member uncovered; and
a tip shield formed of a heat resistant, heat conductive material coaxially surrounding at least a portion of the distal portion of the fiber optic member, the tip shield having an irregular surface structure that is reflective of ultrasonic energy whereby the tip shield enhances reflectivity of ultrasonic energy and enhances heat dissipation as compared to the distal portion of the fiber optic member alone.

2. The laser fiber as claimed in claim 1, in which the fiber optic member is a glass optical fiber having a diameter of about four hundred to about six hundred microns.

3. The laser fiber as claimed in claim 1, in which the tip shield is formed from stainless steel.

4. The laser fiber as claimed in claim 1, in which the tip shield is formed from platinum iridium alloy.

5. The laser fiber as claimed in claim 1, in which the tip shield comprises a coil spring tightly coiled about the distal portion of the fiber optic member.

6. The laser fiber as claimed in claim 1, in which an effective transverse thickness of the tip shield and a longitudinal offset position of the tip shield relative to the distal end are dimensioned such that a line tangent to a shape of distal end at the circumference will intersect the tip shield.

7. The laser fiber as claimed in claim 1, in which the laser fiber has a planar distal face and the tip shield has a distal end that terminates substantially in the same plane as that of the distal planar face.

8. The laser fiber as claimed in claim 1, in which the tip shield tapers so that it is narrower at its distal end than at its proximal end.

9. The laser fiber as claimed in claim 1, in which the tip shield has an outside diameter and the protective jacket has an outside diameter and the tip shield diameter is less than or equal to the protective jacket diameter.

10. The laser fiber as claimed in claim 1, in which the tip shield is formed from nitinol.

11. The laser fiber as claimed in claim 1, in which the tip shield has an outside diameter of about nine hundred fifty microns to about one thousand one hundred microns.

12. An endovenous laser fiber for endovenous laser therapy of varicose veins of the leg, the laser fiber comprising:
means for transmitting light having a proximal end and a distal end the proximal end including a connector adapted to connect to a therapeutic laser and the distal end being adapted to emit laser energy when the means for transmitting light is connected to the therapeutic laser;
means for jacketing the transmitting means covering substantially the entire length of the transmitting means while leaving a portion at the distal end uncovered; and
means for increasing ultrasound reflectivity covering the distal end portion that is uncovered by the jacketing means.

13. The laser fiber as claimed in claim 12, in which the means for increasing ultrasound reflectivity comprises a heat resistant, thermally conductive material.

14. The laser fiber as claimed in claim 12, in which the means for increasing ultrasound reflectivity comprises a material having a thermal conductivity of at least about 12 W/mK at 273 Kelvin.

15. The laser fiber as claimed in claim 12, in which the means for increasing ultrasound reflectivity is formed from metal.

16. The laser fiber as claimed in claim 12, in which the means for increasing ultrasound reflectivity is formed from stainless steel.

17. The laser fiber as claimed in claim 12, in which the means for increasing ultrasound reflectivity is formed from platinum-iridium alloy.

18. The laser fiber as claimed in claim 12, in which the means for increasing ultrasound reflectivity is formed from nitinol.

19. The laser fiber as claimed in claim 12, in which the means for increasing ultrasound reflectivity comprises a coil spring tightly coiled about the distal portion uncovered by the jacket means.

20. The laser fiber as claimed in claim 12, in which the means for increasing ultrasound reflectivity is deflectable upon encountering a blood vessel wall whereby the means for increasing ultrasound reflectivity reduces the tendency of the means for transmitting light from becoming ensnared on the vessel wall.

21. The laser fiber as claimed in claim 12, in which the means for increasing ultrasound reflectivity covers the means for transmitting light substantially to the end thereof whereby the end of the means for transmitting light is prevented from contact with the vessel wall.

23. The laser fiber as claimed in claim 12, in which the means for transmitting light has a planar distal face and the means for increasing ultrasound reflectivity has a distal end that terminates substantially in the same plane as that of the distal planar face.

24. The laser fiber as claimed in claim 12, in which the means for increasing ultrasound reflectivity tapers so that it is narrower at its distal end than at its proximal end.

25. The laser fiber as claimed in claim 12, in which the means for increasing ultrasound reflectivity has an outside diameter and the protective jacket has an outside diameter and the tip shield means for increasing ultrasound reflectivity diameter is less than or equal to the means for jacketing diameter.

26. The laser fiber as claimed in claim 12, in which the means for increasing ultrasound reflectivity is formed from nitinol.

27. The laser fiber as claimed in claim 12, in which the means for increasing ultrasound reflectivity has an outside diameter of about nine hundred fifty microns to about one thousand one hundred microns.

28. A method of improving the ultrasound visibility and heat dissipation characteristics of an end of an endovenous laser fiber for endovenous laser therapy of varicose veins of the leg, the method comprising the steps of:
removing a portion of a jacket from a distal portion of a jacketed fiber optic member; and
applying a tip shield formed of a flexible, heat resistant, heat conductive material coaxially surrounding at least a portion of the distal portion of a fiber optic member from which the jacket has been removed, the tip shield having an irregular surface structure that is reflective of ultrasonic energy.

29. The method as claimed in claim 28, further comprising the step of securing the tip shield to the fiber optic member with a heat resistant adhesive.

30. The method as claimed in claim 28, further comprising the step of the forming the tip shield from stainless steel.

31. The method as claimed in claim 28, further comprising the step of the forming the tip shield from platinum iridium alloy.

32. The method as claimed in claim 28, further comprising the step of the forming the tip shield from nitinol.

33. The method as claimed in claim 28, further comprising the step of the forming the tip shield from a coil spring tightly coiled about the distal portion of the fiber optic member.

34. The method as claimed in claim 28, further comprising the step of the forming the tip shield such that an effective transverse thickness of the tip shield and a longitudinal offset position of the tip shield relative to the distal end are dimensioned such that a line tangent to a shape of distal end at the circumference will intersect the tip shield.

35. The method as claimed in claim 28, in which the laser fiber has a planar distal face and further comprising the step of the forming the tip shield so that a distal end of the tip shield terminates substantially in the same plane as that of the planar distal face.

36. The method as claimed in claim 28, further comprising the step of forming the tip shield tapers so that it is narrower at its distal end than at its proximal end.

37. The method as claimed in claim 28, in which the tip shield has an outside diameter and the jacket has an outside diameter and further comprising the step of forming the tip shield so that its diameter is less than or equal to the jacket diameter.
